(19) United States
(12) Patent Application Publication
(10) Pub. No.: US 2011/0008838 A1
(43) Pub. Date: Jan. 13, 2011

(54) CHIMERIC VARICELLA ZOSTER VIRUS VIRUS-LIKE PARTICLES

(76) Inventors: Gale Smith, Rockville, MD (US); Peter Pushko, Fredereick, MD (US)

Correspondence Address: COOLEY LLP;ATTN: Patent Group, Suite 1100, 777 - 6th Street, NW, WASHINGTON, DC, 20001, US

(21) Appl. No.: 12/669,703

(22) PCT Filed: Jul. 21, 2008
(86) PCT No.: PCT/US08/70636

§ 371 Date: Sep. 10, 2010

Related U.S. Application Data
(60) Provisional application No. 60/950,707, filed on Jul. 19, 2007.
(60) Provisional application No. 60/970,592, filed on Sep. 7, 2007.
(60) Provisional application No. 61/071,835, filed on May 20, 2008.

(51) Int. Cl. C12P 21/02; C07K 14/04; C12N 9/24

(52) U.S. Cl. 435/69.3; 530/403; 435/200

(57) ABSTRACT

The present invention discloses novel chimeric Varicella Zoster Virus (VZV) virus-like particles (VLPs) comprising chimeric VZV glycoproteins. The invention also discloses vaccine formulations of the chimeric VZV-VLPs and methods of inducing an immune response in subjects.

This application claims priority to U.S. applications 60/970,592, filed Sep. 9, 2007, 61/071,835, filed May 20, 2008, and 60/950,707, filed Jul. 19, 2007, all of which are herein incorporated by reference in their entireties for all purposes.

### DESCRIPTION OF THE TEXT FILE SUBMITTED ELECTRONICALLY

The contents of the text file submitted electronically herewith are incorporated herein by reference in their entirety: A computer readable format copy of the Sequence Listing of the Sequence Listing (filename: NOVV 035 02WO SeqList_ST25.txt, date recorded: Jul. 21, 2008, file size 37 kilobytes).

### BACKGROUND OF THE INVENTION

The Varicella Zoster virus (VZV), also known as human herpesvirus 3 (HHV-3), is a member of the alphaherpesvirus subfamily of the Herpesviridae family of viruses. VZV is an enveloped virus with a linear double-stranded DNA genome of approximately 125,000 nucleotides. Its genome is encased by an icosahedral nucleocapsid. The tegument, located in the space between the nucelocapsid and the viral envelope, is a structure comprised of virally-encoded proteins and enzymes. The viral envelope is acquired from host cell membranes and contains viral-encoded glycoproteins.

The VZV genome, the smallest among the human herpesviruses, encodes at least 70 open reading frames, eight of which encode putative glycoproteins (gE, gI, gB, gH, gK, gL, gC, and gM) that function in different steps of the viral replication cycle. Glycoprotein E (gE, also designated ORF 68) is essential for viral replication (Mallory et al. (1997) J. Virol. 71: 8279-8288; Mo et al. (2002) Virology 304: 176-186) and is the most abundant glycoprotein found in infected cells as well as the mature virion (Grose, 2002, The predominant varicella-zoster virus gE and gI glycoprotein complex, In Structure-function relationships of human pathogenic viruses, Holzenburg and Bogner (eds.), Kluwer Academic/Plenum Publishers, New York, N.Y.). Glycoprotein I (gI, also designated ORF 67) forms a complex with gE in infected cells, which facilitates the endocytosis of both glycoproteins and directs them to the trans-Golgi where the final viral envelope is acquired (Olson and Grose (1998) J. Virol. 72:1542-1551). Glycoprotein B (gB, also designated ORF 31), thought to play a role in virus entry, binds to neutralizing antibodies and is the second most prevalent glycoprotein (reviewed in Arvin (1996) Clin. Microbiol. Rev. 9: 361-381). Glycoprotein H (gH) is thought to have a fusion function facilitating cell to cell spread of the virus. Antibodies to gE, gB, and gH are prevalent after natural infection and following vaccination, and have been shown to neutralize viral infectivity in vitro (Keller et al. (1984) J. Virol. 52: 293-297; Arvin et al. (1986) J. Immunol. 137: 1346-1351; Vafai et al. (1988) J. Virol. 62: 2544-2551; Forghani et al. (1990) J. Clin. Microbiol. 28: 2500-2506).

Primary infection with VZV causes chickenpox (varicella) characterized by an extremely contagious skin rash occurring predominantly on the face and trunk. After initial infection, the viral DNA can integrate into the genome of host neuronal cells and remain dormant for several years. The virus can reactivate and cause the disease shingles (herpes zoster) in adults. Shingles produces a skin rash that is distinct from that produced during the primary infection. The rash is associated with severe pain and can result in more serious conditions, such as post-herpetic neuralgia.

A Varicella vaccine (Varivax) is available to the general public and has been added to the recommended vaccination schedule for children in several countries including the United States. A more concentrated formulation of the Varicella vaccine (Zostavax) has been approved by the Food and Drug Administration for prevention of shingles in older members of the population. Although the Varicella vaccine has proven to be safe, there is some evidence that the immunity to VZV infection conferred by the vaccine wanes over time (Chaves et al. (2007) N. Engl. J. Med. 356: 1121-1129). Therefore, vaccinated individuals would remain susceptible to shingles, the more serious condition caused by VZV. In addition, the vaccine is made from live attenuated virus, which creates the possibility of an individual developing either chickenpox or shingles from the vaccination. In fact, there have been several cases of shingles reported that appeared to be caused by the strain used in the vaccine (Matsubara et al. (1995). Acta Paediatr Jpn 37: 648-50; Hammerschlag et al. (1989). J Infect Dis. 160: 535-7). The live attenuated virus present in the vaccine also limits the use of the vaccine in immunocompromised individuals.

Virus-like particles (VLPs) are structurally similar to mature virions, but lack the viral genome making it impossible for viral replication to occur. VLPs contain antigenic proteins, such as capsid and viral envelope proteins, like intact virus and can be constructed to express foreign structural proteins on their surface as well. Therefore, VLPs can be administered safely as an immunogenic composition (e.g. vaccine). Furthermore, since VLPs resemble the native virus and are multivalent particulate structures, VLPs may be more effective in inducing neutralizing antibodies than soluble antigens.

VLPs expressing glycoproteins or tegument proteins have previously been generated from different herpesvirus family members. Light particles (L-particles) comprised of enveloped tegument proteins, have been obtained from cells infected with either herpes simplex virus type 1 (HSV-1), equine herpesvirus type 1 (EHV-1), or pseudorabies virus (McLauchlan and Rixon (1992) J. Gen. Virol. 73: 269-276; U.S. Pat. No. 5,384,122). A different type of VLP, termed pre-viral DNA replication enveloped particles (PREPs), could be generated from cells infected with HSV-1 in the presence of viral DNA replication inhibitors. The PREPs resembled L-particles structurally, but contained a distinct protein composition (Dargan et al. (1995) J. Virol. 69: 4924-4932; U.S. Pat. No. 5,994,116). Hybrid VLPs expressing fragments of the gE protein from VZV have been produced by a technique using protein p1 encoded by the yeast Ty retrotransposon (Garcia-Valcarcel et al. (1997) Vaccine 15: 709-719; Welsh et al. (1999) J. Med. Virol. 59: 78-83; U.S. Pat. No. 6,060,064). The present invention describes novel chimeric VLPs that comprise at least one VZV protein, but does not comprise a yeat Ty protein.

### SUMMARY OF THE INVENTION

This invention comprises a purified chimeric VLP comprising a viral core protein and at least one varicella zoster virus (VZV) protein, wherein said VLP does not comprise a yeast Ty protein and does not comprise VZV nucleic acid. In one embodiment, said VZV protein is chimeric and comprises the ectodomain of a VZV protein and the transmembrane and/or cytoplasmic domain of a heterologous protein. In another embodiment, said heterologous protein associates with said viral core protein. In another embodiment, said VZV protein is selected from the group consisting of gE, gI, gB, gH, gK, gL, gC, and gM. In another embodiment, said VZV protein is gE and/or gI. In another embodiment, said transmembrane and/or cytoplasmic domain of said heterologous protein is from an influenza virus. In another embodiment, said influenza virus protein is hemagglutinin (HA) and/or neuraminidase (NA). In another embodiment, said HA and/or NA is from influenza virus A/Indonesia/5/05. In another embodiment, said viral core is from an orthomyxovirus. In another embodiment, said viral core is from a paramyxovirus. In another embodiment, said chimeric VLP comprises SEQ ID NO. 1 and/or SEQ ID NO. 9.

This invention also comprises a method of producing a chimeric VLP, comprising transfecting a vector encoding at least one VZV protein and a viral core protein into a suitable host cell and expressing said chimeric VZV and viral core protein under conditions that allow VLPs to be formed, wherein said host cell does not comprise a yeast Ty protein. In one embodiment, said VZV protein is chimeric and comprises the ectodomain of a VZV protein and the transmembrane and/or cytoplasmic domain of a heterologous protein. In another embodiment, said VZV protein is gE and/or gI. In another embodiment, said heterologous protein associates with said viral core protein. In another embodiment, said transmembrane and/or cytoplasmic domain of said heterologous protein is from an influenza virus. In another embodiment, said core is influenza virus M1 from influenza virus A/Indonesia/5/05.

This invention also comprises an antigenic formulation comprising VLPs, wherein said chimeric VLPs comprise at least one VZV protein and a viral core protein, and does not comprise a yeast Ty protein. In one embodiment, at least one VZV protein is chimeric and is selected from the group consisting of gE, gI, gB, gH, gK, gL, gC, and gM. In another embodiment, said VZV protein is gE and/or gI. In another embodiment, said VZV protein is gE and/or gI. In another embodiment, said core protein is influenza virus M1 from influenza virus A/Indonesia/5/05.

This invention also comprises a vaccine comprising chimeric VLPs, wherein said chimeric VLPs comprise at least one VZV protein and a viral core protein, and does not comprise a yeast Ty protein. In one embodiment, at least one VZV protein is chimeric and is selected from the group consisting of gE, gI, gB, gH, gK, gL, gC, and gM. In another embodiment, said VZV protein is gE and/or gI. In another embodiment, said VZV protein is gE and/or gI. In another embodiment, said core protein is influenza virus M1 from influenza virus A/Indonesia/5/05 (SEQ ID NO. 9).

This invention also comprises a method of eliciting protective immunity to an infection in a human or animal comprising administering to the human or animal an antigenic formulation or vaccine comprising chimeric VZV-VLPs wherein said chimeric VZV-VLPs comprise at least one VZV protein and a viral core protein, wherein said chimeric VLP does not comprise a yeast Ty protein and does not comprise VZV nucleic acid.

### DESCRIPTION OF THE FIGS.

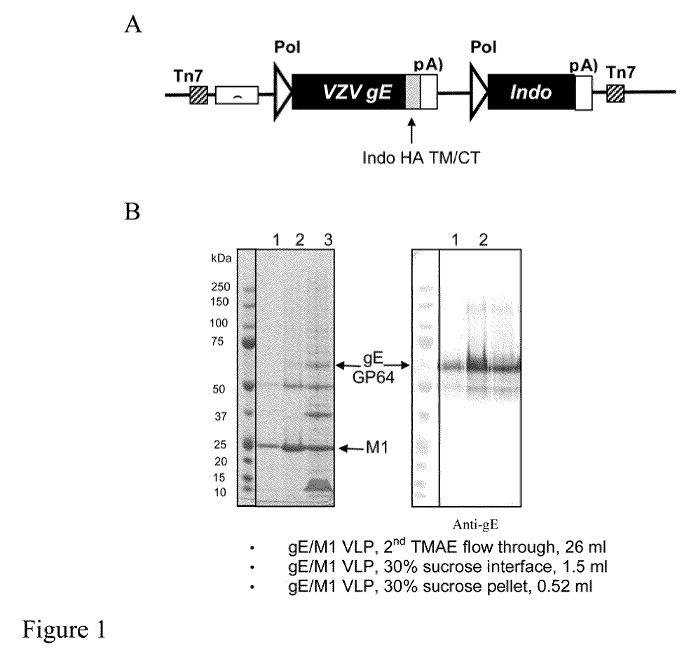

FIG. 1 depicts (A) chimeric VZV gE gene with an influenza transmembrane and C-terminal endodomain from the avian influenza A/Indonesia/5/05 (H5N1) downstream for the AcMNPV baculovirus polyhedrin promoter (PolH) and (B) SDS-PAGE stained with Coomassie blue (left panel) and Western blot with anti-E monoclonal antibody (right panel).

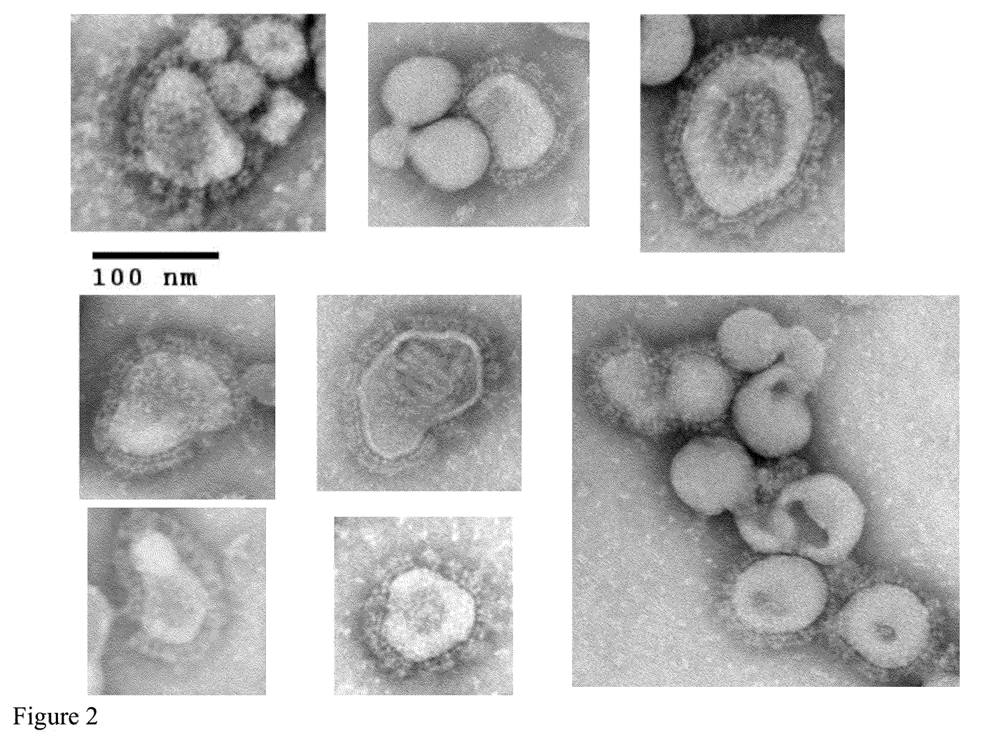

FIG. 2 depicts EM analysis of purified chimeric VZV gE VLPs. Electron microscope (EM) negative stain with PTA of VZV gE(HA TM/CT)/Indo M1 chimeric VLPs (high magnification). The black bar represents 100 nm.

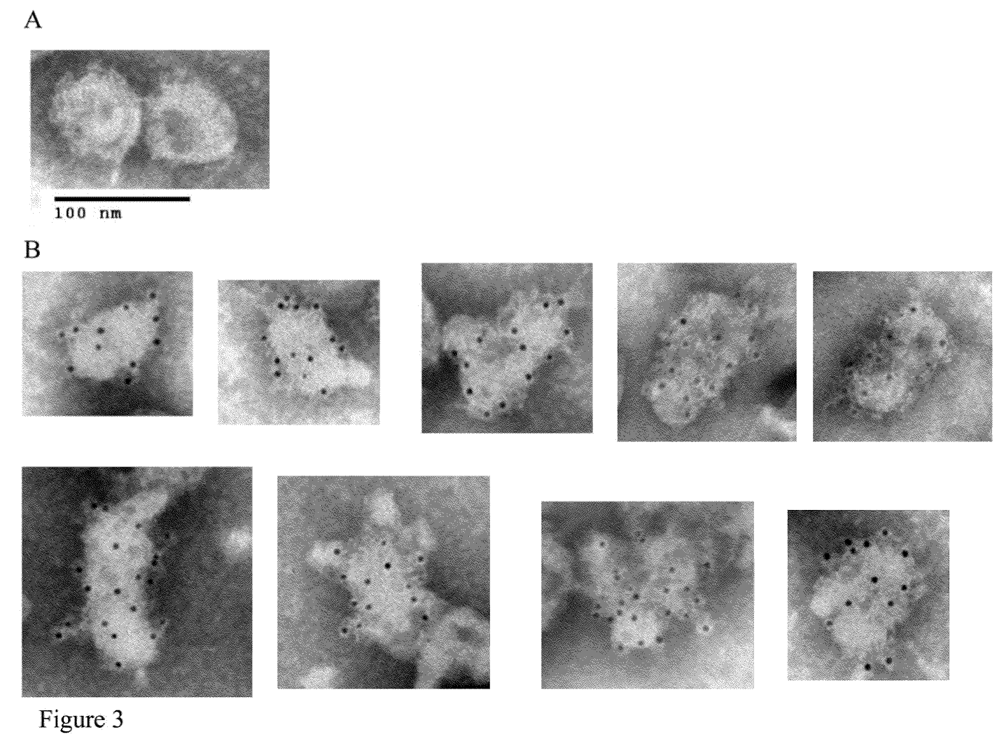

FIG. 3 depicts Immunogold EM analysis of purified chimeric VZV gE VLPs. ImmunoGold EM images of VZV gE (HA TM/CT)/Indo M1 chimeric VLPs. (A) is a control and (B) depicts the 6 nm gold particles located on the chimeric VLPs. The black bar represents 100 nm.

### DETAILED DESCRIPTION

As used herein, the term “virus-like particle” (VLP) refers to a structure that in at least one attribute resembles a virus but which has been demonstrated to be non-infectious. Virus-like particles in accordance with the invention do not carry genetic information encoding for the proteins of the virus-like particles and does not contain a Ty yeast protein, or portion thereof. In general, virus-like particles lack a viral genome and cannot replicate. In addition, virus-like particles can often be produced in large quantities by heterologous expression and can be easily purified. The term also refers to any subviral particle produced by the methods described below. This term includes protein aggregates which can be purified by any of the methods described below or known in the art.

As used herein, the term “chimeric” or “chimeric protein” refers to a protein that comprises a portion of a moiety not produced by VZV. The term excludes a yeast Ty-p1-VZV fusion protein. In general, the term refers to a VZV ectodomain fused to the transmembrane and/or cytoplasmic domain of a heterologous protein.

As used herein, the term “core” protein is a protein that drives budding and release of particles from a host cell. As used herein the terms “core” and “matrix” proteins are used interchangeably. Examples of core proteins that drives budding and release of particles from a host cell comprise influenza M1 and Newcastle disease protein M.

As used herein, the term “antigenic formulation” or “antigenic composition” refers to a preparation which, when administered to a vertebrate, e.g. a mammal, will induce an immune response.

As used herein, the term “purified VLPs” refers to a preparation of VLPs of the invention that is at least 50%, 55% 60%, 65%, 70%, 75%, 80%, 85%, 90%, 95%, 96%, 97%, 98%, 99% or greater, free from other molecules (exclusive of solvent) in a mixture. For example, VLPs of the invention can be substantially free of other viruses, proteins, lipids, and carbohydrates associated with making VLPs of the invention. The term also encompasses VLPs which have been isolated from VLPs which have contaminating VZV genomic DNA or portions thereof.

As used herein, the term “chimeric VLP” refers to VLPs that contain proteins, or portions thereof, from at least two different viruses (heterologous proteins). For the purposes of this invention, one of the proteins is a derived from a virus that can drive the formation of VLPs from host cells (e.g. influenza M1) and the other protein is a chimeric VZV protein.

As used herein, the term “vaccine” refers to a formulation which contains VLPs of the present invention, which is in a form that is capable of being administered to a vertebrate and which induces a protective immune response sufficient to induce immunity to prevent and/or ameliorate an infection and/or to reduce at least one symptom of an infection and/or to enhance the efficacy of another dose of VLPs.

As used herein, the term “effective amount” refers to an amount of VLPs necessary or sufficient to realize a desired biologic effect. An effective amount of the composition would be the amount that achieves a selected result, and such an amount could be determined as a matter of routine by a person skilled in the art. For example, an effective amount for preventing, treating and/or ameliorating an infection could be that amount necessary to cause activation of the immune system, resulting in the development of an antigen specific immune response upon exposure to VLPs of the invention. The term is also synonymous with “sufficient amount.”

As used herein, the term “protective immunity” or “protective immune response” refers to immunity or eliciting an immune response against an infectious agent, which is exhibited by a vertebrate (e.g., a human), that prevents or ameliorates an infection or reduces at least one symptom thereof.

As used herein, the term “vertebrate” or “subject” or “patient” refers to any member of the subphylum cordata, including, without limitation, humans and other primates, including non-human primates such as chimpanzees and other apes and monkey species. Farm animals such as cattle, sheep, pigs, goats and horses; domestic mammals such as dogs and cats; laboratory animals including rodents such as mice, rats (including cotton rats) and guinea pigs; birds, including domestic, wild and game birds such as chickens, turkeys and other gallinaceous birds, ducks, geese, and the like are also non-limiting examples. The terms “mammals” and “animals” are included in this definition. Both adult and newborn individuals are intended to be covered.

In general, virus like particles (VLPs) lack a viral genome and, therefore, are non-infectious. In addition, virus-like particles can often be produced by heterologous expression and can be easily purified. Most VLPs comprise at least a viral core protein. This core protein usually drives budding and release of particles from a host cell. However, for an effective vaccine or antigenic product, antigens should be expressed on VLPs for proper presentation to induce an immune response. This invention comprises chimeric VLPs comprising VZV proteins. In one embodiment, said VZV proteins are chimeric. Chimeric VLPs of the invention are useful for preparing vaccines and immunogenic compositions for treating, preventing and/or ameliorating VZV infection.

One important feature of said chimeric VLPs is the ability to express proteins on the surface of said VLPs so that the immune system of a vertebrate can induce an immune response against said protein. However, not all proteins can be expressed on the surface of VLPs. There may be many reasons why certain proteins are not expressed, or poorly expressed, on the surface of VLPs. One reason is that said protein is not directed to the membrane of a host cell or that said protein does not have a transmembrane domain. Thus, one method of increasing the expression of VZV proteins on the surface of VLPs, and/or increasing the incorporation of said proteins into a VLP, is to fuse the cytoplasmic and/or the transmembrane domain of a heterologous protein to a VZV protein to create a VZV chimeric protein. In one embodiment of the invention, said heterologous cytoplasmic and/or the transmembrane domain may associate with said core protein. Without being bound to a particular theory, said heterologous protein's cytoplasmic and/or the transmembrane domain's association with a core may help drive said chimeric VZV protein into the VLP. Thus, another embodiment of the invention comprises a chimeric VLP, which comprises a core protein that is will drive production of VLP and help drive incorporation of chimeric VZV proteins into the VLP. In another embodiment, said chimeric VLP does not comprise a yeast Ty protein.

In some embodiments, the invention comprises a chimeric VLP comprising at least one chimeric VZV protein, wherein said chimeric VZV protein comprises the transmembrane domain and/or the cytoplasmic domain of a heterologous protein. In one embodiment, said VLP comprises a core protein that drives VLP formation. In another embodiment, said heterologous protein's transmembrane and/or cytoplasmic domain associates with said core protein to drive said chimeric VZV protein into a VLP. In another embodiment, said chimeric VZV protein comprises a portion of a VZV protein selected from the group consisting of gE, gI, gB, gH, gK, gL, gC, and gM. In another embodiment, said VZV protein is gE. In another embodiment, said VZV protein is gI. In another embodiment, chimeric VZV gE and chimeric VZV gI are in the same VLP.

Core proteins that can be used with this invention are viral proteins that when expressed in cells drive VLP formation. In particular, the viral core proteins can include, but are not limited to, a viral Gag protein, in particular, a retrovirus gag protein (e.g. a human immunodeficiency virus (HIV) Gag viral protein (GenBank serial number AAA44987 and p55gag GenBank serial number AAF43628), simian immunodeficiency virus (SIV) Gag viral protein (GenBank serial number AAA91922)), murine leukemia virus (MuLV) Gag viral protein (GenBank serial number NP040332)), a rhabdovirus matrix protein M protein (e.g., a vesicular stomatis virus (VSV) M protein (GenBank accession number CAM83684)), a filovirus viral core protein (e.g., an Ebola VP40 viral protein (GenBank accession number AAN37506)), a Rift Valley Fever virus N protein (GenBank accession number ABK91994), a coronavirus M, E and NP protein (GenBank accession number AAP49024 for NP protein, AAR86790 for M protein, AAP82979 for E protein (SARS coronavirus ShanghaiQXC1)), a bunyavirus N protein (GenBank accession number AAA47114), an influenza M1 protein, a paramyxovirus M protein (e.g., Newcastle disease protein M (AAK55549) and Respiratory syncytial virus (RSV) M (NP_{—}056860)), an arenavirus Z protein (e.g., a Lassa Fever Virus Z protein), Newcastle disease protein M (AAK55549), parainfluenza M (e.g., Human parainfluenza virus 1 MAAB22344)) and combinations thereof. In one embodiment, said core or matrix protein is from an orthomyxovirus. In another embodiment, said orthomyxovirus core does not include influenza virus M1. In another embodiment, said core or matrix protein is from a paramyxovirus. In another embodiment, said paramyxovirus core does not include Newcastle disease virus M, RSV M, or Human parainfluenza virus M.

In another embodiment of the invention, said heterologous proteins may interact with said core proteins to help drive VZV chimeric protein into a VLP. For example if said chimeric VLPs of the invention comprises influenza M1, then said heterologous transmembrane and/or cytoplasmic fused to a VZV protein may be from influenza virus HA and/or NA. These proteins have been shown to help drive heterologous proteins into VLPs (see copending application WO2008/005777, herein incorporated by reference in its entirety). Said core and heterologous protein need not come from the same virus. For example, it has been shown that several transmembrane and/or cytoplasmic domains from non-HIV viruses can be used for expression with p55 gag core from HIV (see Wang et al., J. Virol., 81, 10869-10878, herein incorporated by reference in its entirety).

In certain embodiments, the transmembrane domain and/or cytoplasmic domain of said heterologous protein will be from the same virus. In one embodiment said chimeric VLP comprises influenza virus M1 and chimeric VZV proteins with the transmembrane domain and/or cytoplasmic domain from an influenza HA and/or NA. In another embodiment, said chimeric VLP comprises influenza virus MI from A/Indonesia/5/05 and chimeric VZV proteins with the transmembrane domain and/or cytoplasmic domain from an influenza virus HA and/or NA. In another embodiment, said transmembrane domain and/or cytoplasmic domain from an influenza HA and/or NA is from the group selected from A/quail/Hong Kong/G1/97, A/Hong Kong/1073/99, A/Hong Kong/2108/03, Duck/HK/Y280/97, CK/HK/G9/97, Gf/HK/SSP607/03, Ph/HK/CSW1323/03, WDk/ST/4808/01, CK/HK/NT142/03, CK/HK/WF126/03, SCk/HK/WF285/03, CK/HK/YU463/03, CK/HK/YU577/03, SCk/HK/YU663/03, Ck/HK/CSW161/03, and GF/HK/NT101/03. In another embodiment, the transmembrane domain and/or cytoplasmic domain of influenza virus HA and/or NA is from A/Indonesia/5/05. In another embodiment said chimeric VZV protein comprises the ectodomain of gE with the transmembrane domain and/or cytoplasmic domain of influenza HA and/or NA. In another embodiment, said transmembrane domain of gE is replaced by said influenza HA and/or NA. In one embodiment, said chimeric protein comprises SEQ ID NO. 1. In another embodiment said chimeric VZV protein comprises the ectodomain of gI with the transmembrane domain and/or cytoplasmic domain of influenza virus HA and/or NA. In another embodiment, said chimeric protein comprises SEQ ID NO. 3. In another embodiment, said chimeric VLP comprises an influenza virus M1, a chimeric gE and a chimeric gI. In another embodiment, said chimeric VLP comprises an influenza virus M1, a native gE and a chimeric gI. In another embodiment, said chimeric VLP comprises an influenza virus M1, a chimeric gE and a native gI. In another embodiment, said native gE and Ig has been engineered to have their natural transmembrane domain removed (SEQ IDs 10 and 12, respectively). Since gE and Ig make heterodimers, only one member of said dimer may need to have the heterologous transmembrane and/or cytoplasmic domain for incorporation into VLPs.

In one embodiment of the invention, said chimeric VLP comprises Newcastle disease virus (NDV) protein M and chimeric VZV proteins with the transmembrane domain and/or cytoplasmic domain from a Newcastle disease virus protein. In one embodiment, said transmembrane domain and/or cytoplasmic domain from a NDV protein is from the fusion (F) and/or heamagglutinin-neuraminidase (FIN) protein (Genbank accession number CAA00288 for NDV F and CAA00292 for NDV HN). In another embodiment, said chimeric VLP comprises a NDV M and a chimeric gE. In another embodiment, said chimeric VLP further comprises chimeric gI.

In one embodiment of the invention, said chimeric VLP comprises RSV protein M and chimeric VZV proteins with the transmembrane domain and/or cytoplasmic domain from a RSV protein. In one embodiment, said transmembrane domain and/or cytoplasmic domain from a RSV protein is from the fusion (F) and/or G protein (Genbank accession number AAR 14266 for RSV Fund AAR14265 for RSV G). In another embodiment, said chimeric VLP comprises a RSV M and chimeric gE. In another embodiment, said chimeric VLP further comprises chimeric gI.

In certain embodiments, the transmembrane domain and/or cytoplasmic domain of said heterologous proteins (e.g. influenza HA and/or NA) comprise a spacer sequence between the protein segments. Said space sequences can be any amino acid not native to the VZV or said heterologous protein. This spacer sequence may be important for expressing said VZV chimeric protein on the surface of the VLP. Examples of spacer sequences include poly-G amino acids. Said spacer can be from 1 to about 100 amino acids long.

The invention also encompasses variants of the said VZV proteins expressed on or in the VLPs of the invention (including said chimeras). The variants may contain alterations in the amino acid sequences of the constituent proteins. The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological or immunological activity can be found using computer programs well known in the art, for example, DNASTAR software.

General texts which describe molecular biological techniques, which are applicable to the present invention, such as cloning, mutation, cell culture and the like, include Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (3rd Ed.), Vol. 1-3, Cold Spring Harbor Laboratory, Cold Spring Harbor, N.Y., 2000 (“Sambrook”) and Current Protocols in Molecular Biology, F. M. Ausubel et al., eds., Current Protocols, a joint venture between Greene Publishing Associates, Inc. and John Wiley & Sons, Inc., (“Ausubel”). These texts describe mutagenesis, the use of vectors, promoters and many other relevant topics related to, e.g., the cloning and mutating of gE, gI, gM, gH, gB or tegument proteins of VZV, etc. Thus, the invention also encompasses using known methods of protein engineering and recombinant DNA technology to improve or alter the characteristics of the proteins expressed on or in the chimeric VLPs of the invention. Various types of mutagenesis can be used to produce and/or isolate variant nucleic acids that encode for protein molecules and/or to further modify/mutate the proteins in or on the chimeric VLPs of the invention. They include but are not limited to site-directed, random point mutagenesis, homologous recombination (DNA shuffling), mutagenesis using uracil containing templates, oligonucleotide-directed mutagenesis, phosphorothioate-modified DNA mutagenesis, mutagenesis using gapped duplex DNA or the like. Additional suitable methods include point mismatch repair, mutagenesis using repair-deficient host strains, restriction-selection and restriction-purification, deletion mutagenesis, mutagenesis by total gene synthesis, double-strand break repair, and the like.

Methods of cloning VZV proteins of the invention are known in the art. For example, the gene encoding a specific VZV protein can be isolated by RT-PCR from polyadenylated mRNA extracted from cells which had been infected with a VZV virus. The resulting product gene can be cloned as a DNA insert into a vector. The term “vector” refers to the means by which a nucleic acid can be propagated and/or transferred between organisms, cells, or cellular components. Vectors include plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. In many, but not all, common embodiments, the vectors of the present invention are plasmids.

Thus, the invention comprises nucleotides that encode proteins cloned into an expression vector (e.g. core protein and a least one VZV protein or chimeric VZV protein) that can be expressed in a cell that induces the formation of chimeric VLPs of the invention. An “expression vector” is a vector, such as a plasmid that is capable of promoting expression, as well as replication of a nucleic acid incorporated therein. Typically, the nucleic acid to be expressed is “operably linked” to a promoter and/or enhancer, and is subject to transcription regulatory control by the promoter and/or enhancer. In one embodiment, said nucleotides encode for the VZV gE (ORF 68) protein or chimeric VZV gE protein. In another embodiment, said vector comprises nucleotides that encode the VZV gE and at least one additional VZV protein (e.g. gI or chimeric gI). In another embodiment, said vector comprises nucleotides that encode the VZV gE protein and gI (ORF 67), gM (ORF 50), gH, gB and/or tegument VZV proteins (or chimeric versions of said proteins). In another embodiment, said vector also comprises a core protein capable of driving VLP formation. In another embodiment, the expression vector is a baculovirus vector.

In some embodiments of the invention proteins may comprise, mutations containing alterations which produce silent substitutions, additions, or deletions, but do not alter the properties or activities of the encoded protein or how the proteins are made. Nucleotide variants can be produced for a variety of reasons, e.g., to optimize codon expression for a particular host (change codons in the human mRNA to those preferred by insect cells such as Sf9 cells).

In addition, the nucleotides can be sequenced to ensure that the correct coding regions were cloned and do not contain any unwanted mutations. The nucleotides can be subcloned into an expression vector (e.g. baculovirus) for expression in any cell. The above is only one example of how the VZV viral proteins can be cloned. A person with skill in the art would understand that additional methods are available and are possible.

The invention also provides for constructs and/or vectors that comprise VZV nucleotides that encode for VZV proteins, including gE, gI, gM, gH, gB, tegument proteins, chimeric proteins or portions thereof. The vector may be, for example, a phage, plasmid, viral, or retroviral vector. The constructs and/or vectors that comprise VZV genes, including gE, gI, gM, gH, gB tegument proteins, their chimerics, or portions thereof, should be operably linked to an appropriate promoter, such as the AcMNPV polyhedrin promoter (or other baculovirus), phage lambda PL promoter, the E. coli lac, phoA and tac promoters, the SV40 early and late promoters, and promoters of retroviral LTRs are non-limiting examples. Other suitable promoters will be known to the skilled artisan depending on the host cell and/or the rate of expression desired. The expression constructs will further contain sites for transcription initiation, termination, and, in the transcribed region, a ribosome-binding site for translation. The coding portion of the transcripts expressed by the constructs will preferably include a translation initiating codon at the beginning and a termination codon appropriately positioned at the end of the polypeptide to be translated.

Expression vectors will preferably include at least one selectable marker. Such markers include dihydrofolate reductase, G418 or neomycin resistance for eukaryotic cell culture and tetracycline, kanamycin or ampicillin resistance genes for culturing in E. coli and other bacteria. Among preferred vectors are viral vectors, such as baculovirus, poxvirus (e.g., vaccinia virus, avipox virus, canarypox virus, fowlpox virus, raccoonpox virus, swinepox virus, etc.), adenovirus (e.g., canine adenovirus), herpesvirus, and retrovirus. Other vectors that can be used with the invention comprise vectors for use in bacteria, which comprise pQE70, pQE60 and pQE-9, pBluescript vectors, Phagescript vectors, pNH8A, pNH16a, pNH18A, pNH46A, ptrc99a, pKK223-3, pKK233-3, pDR540, pRIT5. Among preferred eukaryotic vectors are pFastBacI pWINEO, pSV2CAT, pOG44, pXT1 and pSG, pSVK3, pBPV, pMSG, and pSVL. Other suitable vectors will be readily apparent to the skilled artisan.

Next, the recombinant constructs mentioned above could be used to transfect, infect, or transform and can express chimeric VZV proteins comprising gE, gI, gM, gH, gB, and tegument proteins, or portions thereof, and at least one core protein, in eukaryotic cells and/or prokaryotic cells. Thus, the invention provides for host cells which comprise a vector (or vectors) that contain nucleic acids which code for chimeric VZV proteins comprising gE, gI, gM, gH, gB, and tegument proteins, or portions thereof, and at least one core protein, and permit the expression chimeric VZV genes under conditions which allow the formation of VLPs.

Among eukaryotic host cells are yeast, insect, amphibian, avian, plant, C. elegans (or nematode) and mammalian host cells. Non limiting examples of insect cells are, Spodoptera frugiperda (Sf) cells, e.g. Sf9, Sf21, Trichoplusia ni cells, e.g. High Five cells, and Drosophila S2 cells. Examples of fungi (including yeast) host cells are S. cerevisiae, Kluyveromyces lactis (K. lactis), species of Candida including C. albicans and C. glabrala, Aspergillus nidulans, Schizosaccharomyces pombe (S. pombe), Pichia pastoris, and Yarrowia lipolytica. Examples of mammalian cells are COS cells, baby hamster kidney cells, mouse L cells, LNCaP cells, Chinese hamster ovary (CHO) cells, human embryonic kidney (HEK) cells, and African green monkey cells, CV1 cells, HeLa cells, MDCK cells, Vero and Hep-2 cells. Xenopus laevis oocytes, or other cells of amphibian origin, may also be used. Prokaryotic host cells include bacterial cells, for example, E. coli, B. subtilis, and mycobacteria.

Vectors, e.g., vectors comprising polynucleotides of VZV genes (or chimeric VZV genes) which comprise genes that code for gE, gI, gM, gH, gB, and tegument proteins, or portions thereof, and at least one core proteins, can be transfected into host cells according to methods well known in the art. For example, introducing nucleic acids into eukaryotic cells can be by calcium phosphate co-precipitation, electroporation, microinjection, lipofection, and transfection employing polyamine transfection reagents. In one embodiment, said vector is a recombinant baculovirus. In another embodiment, said recombinant baculovirus is transfected into a eukaryotic cell. In a preferred embodiment, said cell is an insect cell. In another embodiment, said insect cell is a Sf9 cell.

In another embodiment, said vector and/or host cell comprise nucleotides that encode chimeric VZV protein gE, or portions thereof. In another embodiment, said vector and/or host cell consists essentially of nucleotides that encode chimeric VZV protein gE, or portions thereof. In a further embodiment, said vector and/or host cell comprise nucleotides that encode chimeric VZV proteins gI, gM, gH, gB, and/or tegument, or portions thereof. In some embodiments, the vectors and/or host cells described above contain chimeric VZV gE, gI, gM, gH, gB, or tegument proteins, or portions thereof, and optionally any additional chimeric VZV proteins, and may contain additional cellular constituents such as cellular proteins, baculovirus proteins, lipids, carbohydrates etc.

The invention also provides for methods of producing chimeric VLPs, said methods comprising expressing at least one core protein and at least one chimeric VZV gene that comprises gE, gI, gM, gH, gB, tegument or portions thereof, under conditions that allow VLP formation. Depending on the expression system and host cell selected, VLPs are produced by growing host cells transformed by an expression vector under conditions whereby the recombinant proteins are expressed and chimeric VLPs are formed. In one embodiment, the invention comprises a method of producing a chimeric VLP, comprising transfecting a vector encoding a viral core protein and a chimeric VZV gE protein into a suitable host cell and expressing said proteins under conditions that allow VLP formation. In another embodiment, the invention comprises a method of producing a chimeric VLP, comprising transfecting a vector encoding a viral core protein, a chimeric VZV gE and a chimeric VZV gI protein into a suitable host cell and expressing said proteins under conditions that allow VLP formation. In another embodiment, said eukaryotic cell is selected from the group consisting of, yeast, insect, amphibian, avian or mammalian cells. In another embodiment, said chimeric VLP does not comprise a yeast Ty protein. The selection of the appropriate growth conditions is within the skill of one of ordinary skill in the art.

Methods to grow cells engineered to produce chimeric VLPs of the invention include, but are not limited to, batch, batch-fed, continuous and perfusion cell culture techniques. Cell culture means the growth and propagation of cells in a bioreactor (a fermentation chamber) where cells propagate and express protein (e.g. recombinant proteins) for purification and isolation. Typically, cell culture is performed under sterile, controlled temperature and atmospheric conditions in a bioreactor. A bioreactor is a chamber used to culture cells in which environmental conditions such as temperature, atmosphere, agitation and/or pH can be monitored.

The chimeric VLPs are then isolated using methods that preserve the integrity thereof, such as by gradient centrifugation, e.g., cesium chloride, sucrose and iodixanol, as well as standard purification techniques including, e.g., ion exchange and gel filtration chromatography. In one embodiment, the invention comprises purified chimeric VLPs of the invention. In another embodiment, said chimeric VLPs of the invention are at least 50%, 55% 60%, 65%, 70%, 75%, 80%, 85%, 90%, 95%, 96%, 97%, 98%, 99% or greater, free from other molecules (exclusive of solvent) present in a mixture. In another embodiment, said chimeric VLPs of the invention are substantially free of other viruses, proteins, lipids, and carbohydrates associated with making chimeric VLPs of the invention. The following is an example of how chimeric VLPs of the invention can be made, isolated and purified. Usually chimeric VLPs are produced from recombinant cell lines engineered to create chimeric VLPs when said cells are grown in cell culture (see above). A person of skill in the art would understand that there are additional methods that can be utilized to make and purify chimeric VLPs of the invention, thus the invention is not limited to the method described.

Production of VLPs of the invention can start by seeding Sf9 cells (non-infected) into shaker flasks, allowing the cells to expand and scaling up as the cells grow and multiply (for example from a 125-ml flask to a 50 Wave bag). The medium used to grow the cell is formulated for the appropriate cell line (preferably serum free media, e.g. insect medium ExCell-420, JRH). Next, said cells are infected with recombinant baculovirus at the most efficient multiplicity of infection (e.g. from about 1 to about 3 plaque forming units per cell). Once infection has occurred, the chimeric VZV protein and/or other proteins self assemble into chimeric VLPs and are secreted from the cells approximately 24 to 72 hours post infection. Usually, infection is most efficient when the cells are in mid-log phase of growth (4-8×10⁶ cells/rill) and are at least about 90% viable.

Chimeric VLPs of the invention can be harvested approximately 48 to 96 hours post infection, when the levels of chimeric VLPs in the cell culture medium are near the maximum but before extensive cell lysis. The Sf9 cell density and viability at the time of harvest can be about 0.5×10⁶ cells/ml to about 1.5×10⁶ cells/ml with at least 20% viability, as shown by dye exclusion assay. Next, the medium is removed and clarified. NaCl can be added to the medium to a concentration of about 0.4 to about 1.0 M, preferably to about 0.5 M, to avoid VLP aggregation. The removal of cell and cellular debris from the cell culture medium containing VLPs of the invention can be accomplished by tangential flow filtration (TFF) with a single use, pre-sterilized hollow fiber 0.5 or 1.00 μm filter cartridge or a similar device.

Next, chimeric VLPs in the clarified culture medium can be concentrated by ultrafiltration using a disposable, pre-sterilized 500,000 molecular weight cut off hollow fiber cartridge. The concentrated VLPs can be diafiltrated against 10 volumes pH 7.0 to 8.0 phosphate-buffered saline (PBS) containing 0.5 M NaCl to remove residual medium components. The concentrated, diafiltered VLPs can be furthered purified on a 20% to 60% discontinuous sucrose gradient in pH 7.2 PBS buffer with 0.5 M NaCl by centrifugation at 6,500×g for 18 hours at about 4° C. to about 10° C. Usually chimeric VLPs will form a distinctive visible band between about 30% to about 40% sucrose or at the interface (in a 20% and 60% step gradient) that can be collected from the gradient and stored. This product can be diluted to comprise 200 mM of NaCl in preparation for the next step in the purification process. This product contains chimeric VLPs and may contain intact baculovirus particles.

Further purification of chimeric VLPs can be achieved by anion exchange chromatography, or 44% isopycnic sucrose cushion centrifugation. In anion exchange chromatography, the sample from the sucrose gradient (see above) is loaded into column containing a medium with an anion (e.g. Matrix Fractogel EMD TMAE) and eluted via a salt gradient (from about 0.2 M to about 1.0 M of NaCl) that can separate the chimeric VLP from other contaminates (e.g. baculovirus and DNA/RNA). In the sucrose cushion method, the sample comprising chimeric VLPs is added to a 44% sucrose cushion and centrifuged for about 18 hours at 30,000 g. VLPs form a band at the top of 44% sucrose, while baculovirus precipitates at the bottom and other contaminating proteins stay in the 0% sucrose layer at the top. The chimeric VLP peak or band is collected.

The intact baculovirus can be inactivated, if desired. Inactivation can be accomplished by chemical methods, for example, formalin or β-propiolactone (BPL). Removal and/or inactivation of intact baculovirus can also be largely accomplished by using selective precipitation and chromatographic methods known in the art, as exemplified above. Methods of inactivation comprise incubating the sample containing the VLPs in 0.2% of BPL for 3 hours at about 25° C. to about 27° C. The baculovirus can also be inactivated by incubating the sample containing the VLPs at 0.05% BPL at 4° C. for 3 days, then at 37° C. for one hour. After the inactivation/removal step, the product comprising VLPs can be run through another diafiltration step to remove any reagent from the inactivation step and/or any residual sucrose, and to place chimeric VLPs into the desired buffer (e.g. PBS). The solution comprising chimeric VLPs can be sterilized by methods known in the art (e.g. sterile filtration) and stored in the refrigerator or freezer.

The above techniques can be practiced across a variety of scales. For example, T-flasks, shake-flasks, spinner bottles, up to industrial sized bioreactors. The bioreactors can comprise either a stainless steel tank or a pre-sterilized plastic bag (for example, the system sold by Wave Biotech, Bridgewater, N.J.). A person with skill in the art will know what is most desirable for their purposes.

### Pharmaceutical or Vaccine Formulations and Administration

The pharmaceutical compositions useful herein contain chimeric VLPs of the invention and a pharmaceutically acceptable carrier, including any suitable diluent or excipient, which includes any pharmaceutical agent that does not itself induce the production of an immune response harmful to the vertebrate receiving the composition, and which may be administered without undue toxicity. As used herein, the term “pharmaceutically acceptable” means being approved by a regulatory agency of the Federal or a state government or listed in the U.S. Pharmacopia, European Pharmacopia or other generally recognized pharmacopia for use in mammals, and more particularly in humans. These compositions can be useful as a vaccine and/or antigenic formulations for inducing a protective immune response in a vertebrate. The invention encompasses an antigenic formulation comprising a chimeric VLP which comprises at least one VZV protein, e.g. chimeric gE protein, but does not include VZV nucleic acid or a yeast Ty protein. In another embodiment, said antigenic formulation comprises a chimeric VLP comprising at least one additional chimeric VZV protein incorporated into the VLP. In another embodiment, said additional chimeric VZV protein comprises gI (ORF 67) protein. In another embodiment, said additional chimeric VZV protein comprises gM (ORF 50) protein. In another embodiment, said additional chimeric VZV protein comprises gH. In another embodiment, said additional chimeric VZV protein comprises gB. In another embodiment, said additional chimeric VZV protein comprises a tegument protein. In another embodiment, said additional chimeric VZV protein comprises a combination of gI, gM, gH, gB or tegument proteins. In another embodiment, said chimeric VZV VLP does not comprise VZV capsid proteins (e.g. ORF 20, ORF 40, ORF 41).

Typically, the vaccine comprises a conventional saline or buffered aqueous solution medium in which the composition of the present invention is suspended or dissolved. In this form, the composition of the present invention can be used conveniently to prevent, ameliorate, or otherwise treat an infection. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses.

The invention also encompasses a vaccine formulation comprising a chimeric VLP which comprises at least one VZV protein, e.g. chimeric gE, but does not include VZV nucleic acid or a yeast Ty protein. In another embodiment, said vaccine formulation comprises a chimeric VLP comprising at least one additional chimeric VZV protein incorporated into the VLP. In another embodiment, said additional chimeric VZV protein comprises chimeric gI (ORF 67) protein. In another embodiment, said additional chimeric VZV protein comprises chimeric gM (ORF 50) protein. In another embodiment, said additional chimeric VZV protein is chimeric gH. In another embodiment, said additional chimeric VZV protein is chimeric gB. In another embodiment, said additional chimeric VZV protein comprises a chimeric tegument protein. In another embodiment, said additional chimeric VZV protein comprises a combination of chimeric gI, gM, gH, gB or tegument proteins. In another embodiment, said VZV VLP does not comprise VZV capsid proteins (e.g. ORF 20, ORF 40, ORF 41).

Said antigenic and vaccine formulations of the invention comprise VLPs of the invention as described above and a pharmaceutically acceptable carrier or excipient. Pharmaceutically acceptable carriers include but are not limited to saline, buffered saline, dextrose, water, glycerol, sterile isotonic aqueous buffer, and combinations thereof. A thorough discussion of pharmaceutically acceptable carriers, diluents, and other excipients is presented in Remington's Pharmaceutical Sciences (Mack Pub. Co. N.J. current edition). The formulation should suit the mode of administration. In a preferred embodiment, the formulation is suitable for administration to humans, preferably is sterile, non-particulate and/or non-pyrogenic.

The pharmaceutical composition, if desired, can also contain minor amounts of wetting or emulsifying agents, or pH buffering agents. The composition can be a solid form, such as a lyophilized powder suitable for reconstitution, a liquid solution, suspension, emulsion, tablet, pill, capsule, sustained release formulation, or powder. Oral formulation can include standard carriers such as pharmaceutical grades of mannitol, lactose, starch, magnesium stearate, sodium saccharine, cellulose, magnesium carbonate, etc.

The invention provides that the chimeric VLP formulation be packaged in a hermetically sealed container such as an ampoule or sachette indicating the quantity of composition. In one embodiment, the chimeric VLP composition is supplied as a liquid, in another embodiment, as a dry sterilized lyophilized powder or water free concentrate in a hermetically sealed container and can be reconstituted, e.g., with water or saline to the appropriate concentration for administration to a subject.

In an alternative embodiment, the chimeric VLP composition is supplied in liquid form in a hermetically sealed container indicating the quantity and concentration of the chimeric VLP composition. Preferably, the liquid form of the chimeric VLP composition is supplied in a hermetically sealed container at least about 50 μg/ml, more preferably at least about 100 μg/ml, at least about 200 μg/ml, at least 500 μg /ml, or at least 1 mg/ml.

Generally, chimeric VZV VLPs of the invention are administered in an effective amount or quantity sufficient to stimulate an immune response against one or more strains of VZV. Preferably, administration of the chimeric VLP of the invention elicits immunity against VZV. Typically, the dose can be adjusted within this range based on, e.g., age, physical condition, body weight, sex, diet, time of administration, and other clinical factors. The prophylactic vaccine formulation is systemically administered, e.g., by subcutaneous or intramuscular injection using a needle and syringe, or a needle-less injection device. Alternatively, the vaccine formulation is administered intranasally, either by drops, large particle aerosol (greater than about 10 microns), or spray into the upper respiratory tract. While any of the above routes of delivery results in an immune response, intranasal administration confers the added benefit of eliciting mucosal immunity at the site of entry of many viruses, including VZV.

Thus, the invention also comprises a method of formulating a vaccine or antigenic composition that induces immunity to an infection or at least one symptom thereof to a mammal, comprising adding to said formulation an effective dose of a chimeric VZV VLP. In one embodiment, said infection is a VZV infection. An “effective dose” generally refers to that amount of VLPs of the invention sufficient to induce immunity, to prevent and/or ameliorate an infection or to reduce at least one symptom of an infection and/or to enhance the efficacy of another dose of a chimeric VLP. An effective dose may refer to the amount of chimeric VLPs sufficient to delay or minimize the onset of an infection. An effective dose may also refer to the amount of chimeric VLPs that provide a therapeutic benefit in the treatment or management of an infection. Further, an effective dose is the amount with respect to chimeric VLPs of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

As mentioned above, the VLPs of the invention prevent or reduce at least one symptom of VZV infection in a subject. Symptoms of the two diseases caused by VZV infection are well known in the art. Symptoms of chickenpox (varicella), produced by primary VZV infection, include fever, malaise, headache, abdominal pain, fatigue, anorexia, and skin lesions occurring predominantly on the scalp, face, and trunk. Shingles (herpes zoster), resulting from a reactivation of the latent VZV, is characterized by the following symptoms: a skin rash usually appearing unilaterally in a thoracic dermatome, acute neuritic pain, and hypersensitivity. Thus, the method of the invention comprises the prevention or reduction of at least one symptom associated with VZV infection. A reduction in a symptom may be determined subjectively or objectively, e.g., self assessment by a subject, by a clinician's assessment or by conducting an appropriate assay or measurement (e.g. body temperature), including, e.g., a quality of life assessment, a slowed progression of a VZV infection or additional symptoms, a reduced severity of VZV symptoms or a suitable assays (e.g. antibody titer and/or T-cell activation assay). The objective assessment comprises both animal and human assessments.

While stimulation of immunity with a single dose is preferred, additional dosages can be administered, by the same or different route, to achieve the desired effect. In neonates and infants, for example, multiple administrations may be required to elicit sufficient levels of immunity. Administration can continue at intervals throughout childhood, as necessary to maintain sufficient levels of protection against infections, e.g. VZV infection. Similarly, adults who are particularly susceptible to repeated or serious infections, such as, for example, health care workers, day care workers, family members of young children, the elderly, and individuals with compromised cardiopulmonary function may require multiple immunizations to establish and/or maintain protective immune responses. Levels of induced immunity can be monitored, for example, by measuring amounts of neutralizing secretory and serum antibodies, and dosages adjusted or vaccinations repeated as necessary to elicit and maintain desired levels of protection.

Methods of administering a composition comprising chimeric VLPs (vaccine and/or antigenic formulations) include, but are not limited to, parenteral administration (e.g., intradermal, intramuscular, intravenous and subcutaneous), epidural, and mucosal (e.g., intranasal and oral or pulmonary routes or by suppositories). In a specific embodiment, compositions of the present invention are administered orally, intradermally, intranasally, intramuscularly, intraperitoneally, intravenously, or subcutaneously. The compositions may be administered by any convenient route, for example by infusion or bolus injection, by absorption through epithelial or mucocutaneous linings (e.g., oral mucous, colon, conjunctiva, nasopharynx, oropharynx, vagina, urethra, urinary bladder and intestinal mucosa, etc.) and may be administered together with other biologically active agents. In some embodiments, intranasal or other mucosal routes of administration of a composition comprising chimeric VLPs of the invention may induce an antibody or other immune response that is substantially higher than other routes of administration. In another embodiment, intranasal or other mucosal routes of administration of a composition comprising chimeric VLPs of the invention may induce an antibody or other immune response that will induce cross protection against other strains of VZV. Administration can be systemic or local.

Vaccines and/or antigenic formulations of the invention may also be administered on a dosage schedule, for example, an initial administration of the vaccine composition with subsequent booster administrations. In particular embodiments, a second dose of the composition is administered anywhere from two weeks to one year, preferably from about 1, about 2, about 3, about 4, about 5 to about 6 months, after the initial administration. Additionally, a third dose may be administered after the second dose and from about three months to about two years, or even longer, preferably about 4, about 5, or about 6 months, or about 7 months to about one year after the initial administration. The third dose may be optionally administered when no or low levels of specific immunoglobulins are detected in the serum and/or urine or mucosal secretions of the subject after the second dose. In a preferred embodiment, a second dose is administered about one month after the first administration and a third dose is administered about six months after the first administration. In another embodiment, the second dose is administered about six months after the first administration. In another embodiment, said chimeric VLPs of the invention can be administered as part of a combination therapy. For example, chimeric VLPs of the invention can be formulated with other immunogenic compositions, antivirals and/or antibiotics.

The dosage of the pharmaceutical formulation can be determined readily by the skilled artisan, for example, by first identifying doses effective to elicit a prophylactic or therapeutic immune response, e.g., by measuring the serum titer of virus specific immunoglobulins or by measuring the inhibitory ratio of antibodies in serum samples, or urine samples, or mucosal secretions. Said dosages can be determined from animal studies. A non-limiting list of animals used to study the efficacy of vaccines include the guinea pig, hamster, ferrets, chinchilla, mouse and cotton rat. Most animals are not natural hosts to infectious agents but can still serve in studies of various aspects of the disease. For example, any of the above animals can be dosed with a vaccine candidate, e.g. chimeric VLPs of the invention, to partially characterize the immune response induced, and/or to determine if any neutralizing antibodies have been produced. For example, many studies have been conducted in the mouse model because mice are small size and their low cost allows researchers to conduct studies on a larger scale.

In addition, human clinical studies can be performed to determine the preferred effective dose for humans by a skilled artisan. Such clinical studies are routine and well known in the art. The precise dose to be employed will also depend on the route of administration. Effective doses may be extrapolated from dose-response curves derived from in vitro or animal test systems.

As also well known in the art, the immunogenicity of a particular composition can be enhanced by the use of non-specific stimulators of the immune response, known as adjuvants. The term “adjuvant” refers to a compound that, when used in combination with a specific immunogen (e.g. a VLP) in a formulation, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. Adjuvants have been used experimentally to promote a generalized increase in immunity against unknown antigens (e.g., U.S. Pat. No. 4,877,611). Immunization protocols have used adjuvants to stimulate responses for many years, and as such, adjuvants are well known to one of ordinary skill in the art. Some adjuvants affect the way in which antigens are presented. For example, the immune response is increased when protein antigens are precipitated by alum. Emulsification of antigens also prolongs the duration of antigen presentation. The inclusion of any adjuvant described in Vogel et al., “A Compendium of Vaccine Adjuvants and Excipients (2^{nd} Edition),” herein incorporated by reference in its entirety for all purposes, is envisioned within the scope of this invention.

Exemplary, adjuvants include complete Freund's adjuvant (a non-specific stimulator of the immune response containing killed Mycobacterium tuberculosis), incomplete Freund's adjuvants and aluminum hydroxide adjuvant. Other adjuvants comprise GMCSP, BCG, aluminum hydroxide, MDP compounds, such as thur-MDP and nor-MDP, CGP (MTP-PE), lipid A, and monophosphoryl lipid A (MPL). RIBI, which contains three components extracted from bacteria, MPL, trehalose dimycolate (TDM) and cell wall skeleton (CWS) in a 2% squalene/Tween 80 emulsion also is contemplated. MF-59, Novasomes®, MHC antigens may also be used.

In one embodiment of the invention, the adjuvant is a paucilamellar lipid vesicle having about two to ten bilayers arranged in the form of substantially spherical shells separated by aqueous layers surrounding a large amorphous central cavity free of lipid bilayers. Paucilamellar lipid vesicles may act to stimulate the immune response several ways, as non-specific stimulators, as carriers for the antigen, as carriers of additional adjuvants, and combinations thereof. Paucilamellar lipid vesicles act as non-specific immune stimulators when, for example, a vaccine is prepared by intermixing the antigen with the preformed vesicles such that the antigen remains extracellular to the vesicles. By encapsulating an antigen within the central cavity of the vesicle, the vesicle acts both as an immune stimulator and a carrier for the antigen. In another embodiment, the vesicles are primarily made of nonphospholipid vesicles. In other embodiment, the vesicles are Novasomes®. Novasomes® are paucilamellar nonphospholipid vesicles ranging from about 100 nm to about 500 nm. They comprise Brij 72, cholesterol, oleic acid and squalene. Novasomes have been shown to be an effective adjuvant for influenza antigens (see, U.S. Pat. Nos. 5,629,021, 6,387,373, and 4,911,928, herein incorporated by reference in their entireties for all purposes).

The chimeric VLPs of the invention can also be formulated with “immune stimulators.” The term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as VLPs of the invention, or can be administered separately. Either the protein or an expression vector encoding the protein can be administered to produce an immunostimulatory effect. Thus in one embodiment, the invention comprises antigenic and vaccine formulations comprising an adjuvant and/or an immune stimulator.

This invention is further illustrated by the following examples that should not be construed as limiting. The contents of all references, patents and published patent applications cited throughout this application, as well as the Figures and the Sequence Listing, are incorporated herein by reference for all purposes.

### Examples

### Example 1

Described below is the cloning, expression and purification process of chimeric VZV VLPs comprising chimeric gE (HA TM/CT) glycoprotein (SEQ ID No. 1) and the avian influenza M1 (A/Indonedia/5/05 H5N1 strain) matrix protein (SEQ ID No. 9) in Sf9 insect cells. Chimeric gE and avian influenza M1 were cloned and expressed in a baculovirus expression system under conditions that allow for VLP formation. This construct is depicted in FIG. 1A.

To confirm that VLPs were made, Sf9 cell culture medium containing the Sf9 cells infected baculovirus comprising VZV gE (HA TM/CT)/Indo M1 chimeric construct was centrifuged by low speed centrifugation 64 hours post baculovirus infection. The cell free medium was concentrated by ultrafiltration (UF) with a 500 kDa MWCO hollow fiber filter (GE healthcare). The retentate was buffer exchanged with diafiltration (DF) to 25 mM TrisCl pH 8.0, 500 mM NaCl. The UF/DF retentate was loaded on an ion exchange column (Fractogel TMAE) equilibrated in the same buffer.

Next, the flow through fractions containing VLPs and baculovirus were further concentrated with ultrafiltration before loading onto a Sephacryl S500 size exclusion column (GE healthcare) equilibrated with 25 mM TrisCl pH 8.0 300 mM NaCl. VLPs and baculovirus fractions were eluted in the void volume of the S500 column while soluble proteins and cell fragments were eluted in later chromatography fractions. The void peak from S500 column was loaded to a 2nd TMAE column at 300 mM NaCl. Under these conditions, VLPs do not bind to the anionic resin while more negatively charged baculovirus bind to the column. This was a critical separation step which relies on the fact that VLPs are devoid of a DNA or RNA genome while baculoviruses contains a large 137 KDa double stranded DNA genome which increases the negative charge of the baculovirus and thus the binding characteristics of the virus. The 2nd TMAE flow through fractions containing VLPs were concentrated with ultracentrifugation (100 K g hour) through a 30% sucrose cushion (FIG. 4B, lane 2). Next the purified chimeric VZV gE(HA TM/CT)/Indo M1 chimeric VLPs were used for SDS PAGE analysis, western blot analyses, electron microscope(EM) and immunogold analysis.

The SDS PAGE and western blot analysis were performed as previously described. These results are shown in FIG. 1B. Lane 1 are VLPs from the second TMAE ion exchange chromatography column and lanes 2 and 3 are VLPs recovered near the top of a 30% sucrose interface and the pellet, respectively after high speed centrifugation. Note that trace amounts of baculovirus and VZV chimeric VLPs were pelleted by the ultracentrifugation but the majority of gE/M1 chimeric VLPs were a higher buoyant density and near the 30% sucrose interface. Note that chimeric gE (HA TM/CT) has the same molecular weight as baculovirus protein GP64, on a gel the bands overlap. Thus, the 64 kDa protein in lane 2 (comprising purified VLPs) is mainly chimeric gE (HA TM/CT) and the same size band in lane 3 (comprising VLPs and baculovirus) is primarily the baculovirus envelope glycoprotein GP64. This conclusion was drawn by comparison of the Coomassie blue stained gel and anti-gE Western blot. Purified chimeric VZV VLPs (lane 1) were >90% pure with the major proteins being M1 and the M1 dimmer which is not fully disrupted by SDS (25 KDa and 50 KDa) and the chimeric gE (60 KDa). Thus, these results show that chimeric VLPs were produced and purified.

Next, the chimeric VLPs were imaged with electron microscopy (EM) negative stain (FIG. 2). These EM images shows that chimeric VLPs have a spherical core with a highly structured envelop.

Immunogold staining of the VLPs were performed to determine if VZV gE was expressed on the surface of chimeric VLPs. Before the EM images were taken, the chimeric VLPs were either incubated with normal mouse IgG (A) or mouse anti-VZV-gE (clone 9C8 from Santa Cruz) (B) followed by incubation with gold labeled secondary antibody against mice. The 6 nm gold particles located on the chimeric VLPs confirmed the presence of gE protein on VLPs. As shown is FIG. 3, the antibodies did stain the VLPs. Thus, the VLPs do express VZV gE.

These data show that chimeric VZV VLPs comprising chimeric VZV proteins can be produced and purified.

All patents, publications and patent applications herein are incorporated by reference to the same extent as if each individual patent, publication or cited patent application was specifically and individually indicated to be incorporated by reference.

The foregoing detailed description has been given for clearness of understanding only and no unnecessary limitations should be understood therefrom as modifications will be obvious to those skilled in the art. It is not an admission that any of the information provided herein is prior art or relevant to the presently claimed inventions, or that any publication specifically or implicitly referenced is prior art.

Unless defined otherwise, all technical and scientific terms used herein have the same meaning as commonly understood by one of ordinary skill in the art to which this invention belongs.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1.-50. (canceled)

51. A virus-like particle (VLP) comprising an influenza matrix (M1) protein and at least one Varicella Zoster Virus (VZV) protein.

52. The VLP of claim 51, wherein said influenza M1 protein is derived from an avian influenza virus.

53. The VLP of claim 52, wherein said avian influenza virus is A/Indonesia/5/05.

54. The VLP of claim 53, wherein said M1 protein comprises SEQ ID NO: 9.

55. The VLP of claim 51, wherein said VZV protein is selected from the group consisting of gE, gI, gB, gH, gK, gL, gC, gM, and tegument.

56. The VLP of claim 52, wherein said VZV protein is selected from the group consisting of gE, gI, gB, gH, gK, gL, gC, gM, and tegument.

57. The VLP of claim 55, wherein said VZV protein is gE.

58. The VLP of claim 57, wherein said VLP further comprises gI.

59. The VLP of claim 58, wherein said VLP further comprises tegument.

60. The VLP of claim 56, wherein said VZV protein is gE.

61. The VLP of claim 60, wherein said VLP further comprises gI.

62. The VLP of claim 61, wherein said VLP further comprises tegument.

63. The VLP of claim 51, wherein said VZV protein is chimeric and comprises the ectodomain of a VZV protein and the transmembrane and/or cytoplasmic domain of a heterologous protein.

64. The VLP of claim 63, wherein said transmembrane and/or cytoplasmic domain of a heterologous protein is from an influenza virus.

65. The VLP of claim 64, wherein said influenza virus protein is hemagglutinin (HA) and/or neuraminidase (NA).

66. The VLP of claim 65, wherein said HA and/or said NA is derived from A/Indonesia/5/05.

67. The VLP of claim 51, wherein said VLP comprises SEQ ID NO: 1 and SEQ ID NO: 9.

68. An antigenic formulation comprising the VLP of claim 51.

69. A method of producing a VLP, comprising transfecting one or more vectors encoding at least one VZV protein and an influenza matrix (M1) protein into a suitable host cell and expressing said VZV protein and said influenza matrix (M1) protein under conditions that allow VLPs to be formed.

70. The method of claim 69, wherein said influenza M1 protein is derived from an avian influenza virus.
